(19) United States
(12) Patent Application Publication
(10) Pub. No.: US 2014/0378766 A1
(43) Pub. Date: Dec. 25, 2014

(54) ENDOTRACHEAL INTUBATION ASSISTANCE APPARATUS

(71) Applicant: Gentle and Tender Co., LTD., Taipei City (TW)

(72) Inventor: Po-Han Lo, Taipei City (TW)

(73) Assignee: Gentle and Tender Co., LTD., Taipei City (TW)

(21) Appl. No.: 14/481,197

(22) Filed: Sep. 9, 2014

Related U.S. Application Data
(63) Continuation-in-part of application No. 13/793,039, filed on Mar. 11, 2013.

(30) Foreign Application Priority Data
Mar. 13, 2012 (TW) 101108479
Sep. 11, 2013 (TW) 102132770

(51) Int. Cl. A61M 16/04; A61B 1/00; A61B 1/005; A61B 1/267

(52) U.S. Cl. CPC A61M 16/0488; A61B 1/267; A61B 1/00066; A61B 1/0055; A61B 1/00133; A61M 2210/0643
USPC 600/120

(57) ABSTRACT

An endotracheal intubation assistance apparatus is for assisting in insertion of an endotracheal tube into the trachea of a patient, and includes a flexible movable tubular stylet, a graspable controller, and a viewing device. The stylet has a leading section, a body section, a tail section, and two slits extending through the body section and the tail section for dividing the tail section into first and second driven sheets. The viewing device includes an elongate body and a viewing head. The elongate body and the viewing head are movable through the controller, and is extendable outwardly from the leading section. When the first and second driven sheets move relative to each other, the leading section swings synchronously a distal end of the endotracheal tube and the viewing head.

### CROSS-REFERENCE TO RELATED APPLICATION

This application claims priority of Taiwanese patent application no. 102132770, filed on Sep. 11, 2013, and is a continuation-in-part application of U.S. patent application Ser. No. 13/793,039, filed on Mar. 11, 2013 and claiming priority of Taiwanese Application No. 101108479, which was filed on Mar. 13, 2012.

### BACKGROUND OF THE INVENTION

1. Field of the Invention

This invention relates to a medical instrument, and more particularly to an endotracheal intubation assistance apparatus that has a simple structure, a low cost, and a high clinical applicability.

2. Description of the Related Art

It is important for endotracheal intubation to be performed in a safety and quick manner. Otherwise, teeth or laryngeal tissue of the patient may be hurt, or an endotracheal tube is inserted erroneously into the esophagus disposed behind the trachea, thereby resulting in occurrence of severe complications, such as hypoxia.

Referring to FIG. 1, a conventional endotracheal tube is associated with a standard stylet 20. The endotracheal tube 10 includes a tube body 101 having a predetermined curvature and flexibility, a distal end 102, a proximal end 103, a connector 104 sleeved on the proximal end 103, and an inflatable cuff 105 adjacent to the distal end 102. The standard stylet 20 can be shaped manually to change the curvature of the endotracheal tube 10 (i.e., the angle of the distal end 102). When the angle is not correct, it is necessary to draw an assembly of the endotracheal tube 10 and the standard stylet 20 out of the mouth of the patient for shape adjustment. Upon the completion of the shape adjustment to align the distal end 102 with the vocal cords of the patient, the endotracheal tube 10 is inserted into the trachea.

The standard stylet 20 may be replaced with a bougie having a greater length, or a lightwand, as disclosed in US NO. 20080017195. A leading end of the bougie or the lightwand is inserted into the trachea. Under guide of the bougie or the lightwand, the endotracheal tube can be pass fully through a space between the vocal cords. However, the medical personnel must have the related experience and skill. Furthermore, such an endotracheal intubation device may not be suitable for patients more difficult to intubate.

U.S. Pat. Nos. 3,802,440, 4,949,716, 5,259,377, and 5,791,338 disclose various auxiliary devices for adjusting the curvature of the endotracheal tube, each of which includes a movable stylet and a control mechanism for activating the movable stylet in a manner more efficient than the above-mentioned manual shaping manner.

To perform the above intubation procedures, a blade of a laryngoscope (e.g., disclosed in U.S. Pat. No. 3,638,644) is required to press against the tongue of the patient for allowing light to be emitted into the throat of the patient. However, if the mouth of the patient cannot open to a larger extent, it is difficult to put the laryngoscope into the mouth. Or, although the laryngoscope can be inserted into the mouth, the throat of the patient cannot be seen clearly by the medical personnel. In this case, blind intubation may be tried.

U.S. Pat. Nos. 3,669,098, 5,327,881, 6,319,195, 6,539,942, 7,458,375, and US Pub. No. 2008/0236575 disclose a viewing device that is built in a movable stylet. The viewing device is configured as an optical fiber assembly, small video camera, or bronchoscope (e.g., fiber bronchoscope or video bronchoscope), and is used with an ocular or display for showing images. This can eliminate use of a bulky laryngoscope to reduce anatomical distortion, and can perform real time observation of the vocal cords of the patient. In this manner, leading ends of the movable stylet and the endotracheal tube can be moved into the proximity of the vocal cords, followed by inserting only the endotracheal tube into the trachea. However, the endotracheal tube has a relatively large outer diameter so that, when the space between the vocal cords is small, when anatomical distortion or the throat swelling occurs, or when the throat reflex is triggered by external stimulation, it is difficult to intubate fully. Furthermore, if the angle formed between the longitudinal directions of the distal end of the endotracheal tube and the trachea is too large, it is more difficult to intubate fully.

Since the movable stylet typically has a complex structure, and cooperates with the viewing device to form one piece, such a one piece structure can not be designed to be disposable.

To overcome the blind condition of the bougie or the lightwand, U.S. Pat. No. 6,978,784 and US Pub. No. 2007/0175482 disclose a viewing device that permits an endotracheal tube to be sleeved thereon and that can serve as a guide device. The viewing device is first inserted into the trachea. Subsequently, the endotracheal tube is also inserted into the trachea along the viewing device. For awake and obedient patients, such an endotracheal intubation process has been considered to be a current gold standard. However, the stiffness and maneuverability of the viewing device are not sufficient, so that the skill standard of the medical personnel for performing the endotracheal intubation process is relatively high. Since the hardness of the optic fiber assembly is not sufficient, before the optic fiber assembly is inserted into the trachea of a patient, a front end thereof may twist or swing leftwardly or rightwardly due to interference of a soft tissue (such as a tongue or an epiglottis), so that images in the display cannot be indentified. That is, a disorientation problem is encountered. U.S. Pat. No. 6,257,236 discloses a bronchoscope for facilitating insertion of a stylet into the trachea to allow an endotracheal tube to be sleeved on the stylet and then pushed into the trachea. However, the stiffness and maneuverability of the bronchoscope are also not sufficient. U.S. Pat. No. 6,508,757 discloses a malleable material that is sleeved on a viewing device to increase the stiffness of the viewing device. However, an assembly of the viewing device and the malleable material needs to be frequently inserted into and removed from the mouth for angle adjustment, as required by the standard stylet. U.S. Pat. No. 6,146,402 discloses a guide tube introducer for facilitating a guide wire to be placed into the trachea. However, the guide tube introducer does not have bendability to result in insufficient maneuverability, and cannot be preloaded with an endotracheal tube due to the fact that the guide tube introducer must be removed prior to mounting the endotracheal tube.

### SUMMARY OF THE INVENTION

The object of this invention is to provide an endotracheal intubation assistance apparatus that has a simple structure, a low cost, and a high clinic applicability.

According to this invention, there is provided an endotracheal intubation assistance apparatus adapted for assisting in insertion of an endotracheal tube into the trachea of a patient, the endotracheal tube including a flexible tube body having a predetermined curvature, a distal end, and a proximal end, the endotracheal intubation assistance apparatus comprising:

a movable tubular stylet being flexible and having a leading section, a tail section, a body section connected between the leading section and the tail section along length of the movable tubular stylet, and two slits extending through the body section and the tail section, the leading section being configured as a cylinder and having an inner bore, the body section being divided by the slits into a first strip and a second strip, the tail section being divided by the slits into a first driven sheet connected to the first strip, and a second driven sheet connected to the second strip, the leading section and the body section being adapted to extend in the tube body of the endotracheal tube;

a graspable controller including a main body, a driving mechanism disposed on the main body, and an operating member operable for activating the driving mechanism, the main body being adapted to be connected to the proximal end of the endotracheal tube and permitting the tail section of the movable tubular stylet to extend thereinto, the driving mechanism being drivable to move first and second driven sheets relative to each other along the length of the movable tubular stylet; and

a viewing device including a flexible elongate body, a viewing head disposed on a leading end of the elongate body, and a multi-joint disposed in the elongate body, the elongate body and the viewing head being movable in the graspable controller and being adapted to be extendable outwardly from the inner bore in the leading section of the movable tubular stylet such that, when the first and second driven sheets are driven to move relative to each other along the length of the movable tubular stylet, the leading section can swing the distal end of the endotracheal tube and the viewing head synchronously therewith, the multi-joint link having a plurality of joint bodies that are interconnected in series and that have rotating axes generally parallel to each other.

By controlling relative movement between the first and second driven sheets, the leading section can swing the distal end of the endotracheal tube and the viewing head synchronously therewith, so as to allow the elongate body to move toward the trachea by a predetermined distance for guiding the endotracheal tube to move toward the trachea along the elongate body. In this manner, the success rate of endotracheal intubation can be promoted effectively, and intubation time period can be reduced.

### BRIEF DESCRIPTION OF THE DRAWINGS

These and other features and advantages of this invention will become apparent in the following detailed description of an embodiment of this invention, with reference to the accompanying drawings, in which:

FIG. 1 is a schematic side view of a conventional assembly of an endotracheal tube and a standard stylet;

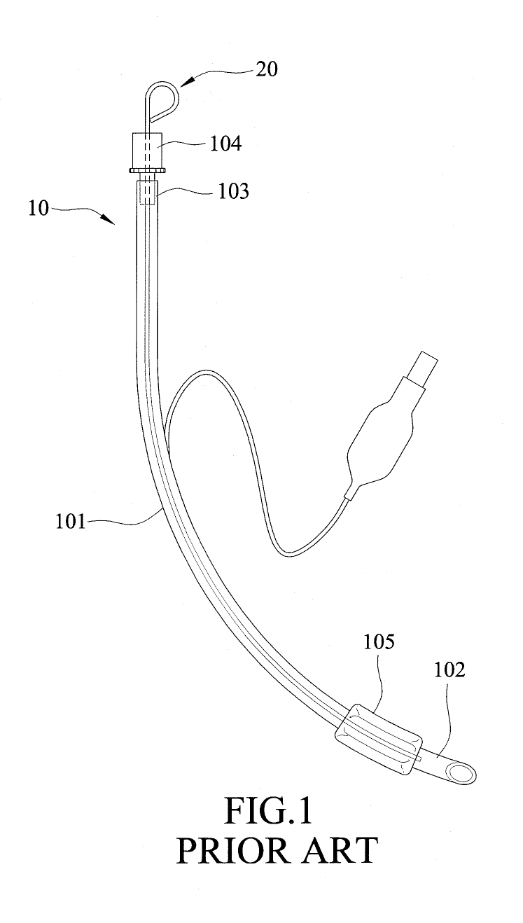

FIG. 2 is a schematic view of the embodiment of an endotracheal intubation assistance apparatus according to this invention, illustrating that a leading section of a movable tubular stylet and a distal end of an endotracheal tube are located adjacent to the vocal cords;

FIG. 3 is a perspective view of the movable tubular stylet of the embodiment;

FIG. 4 is a fragmentary exploded perspective view of the embodiment, illustrating a graspable controller;

FIG. 5 is a perspective view of the graspable controller of the embodiment;

FIG. 6 is a sectional view taken along line VI-VI in FIG. 5;

FIG. 7 is a sectional view taken along line VII-VII in FIG. 6;

FIG. 8 is a schematic perspective view of a viewing device of the embodiment, illustrating an elongate body, a viewing head, a multi-joint link, and a pin;

FIG. 9 is a fragmentary schematic sectional view of the embodiment, illustrating a locking, position of a tongue plate of the graspable controller;

FIG. 10 is a view similar to FIG. 9 but illustrating a release position of the tongue plate;

FIG. 11 is a schematic view of the embodiment, illustrating operation of an operating member of the graspable controller and the movable tubular stylet;

FIG. 12 is a schematic view of the embodiment, illustrating operation of the graspable controller and the multi-joint link of the viewing device;

FIG. 13 is a sectional view taken along line XIII-XIII in FIG. 11;

FIG. 14 is a schematic view of the embodiment, illustrating that the elongate body of the viewing device is inserted into the endotracheal tube;

FIG. 15 is a schematic view of the embodiment, illustrating that the endotracheal tube and the movable tubular stylet are inserted into the trachea along the elongate body; and

FIG. 16 is a schematic view of the embodiment, illustrating that only the endotracheal tube is left within the trachea.

### DETAILED DESCRIPTION OF THE EMBODIMENT

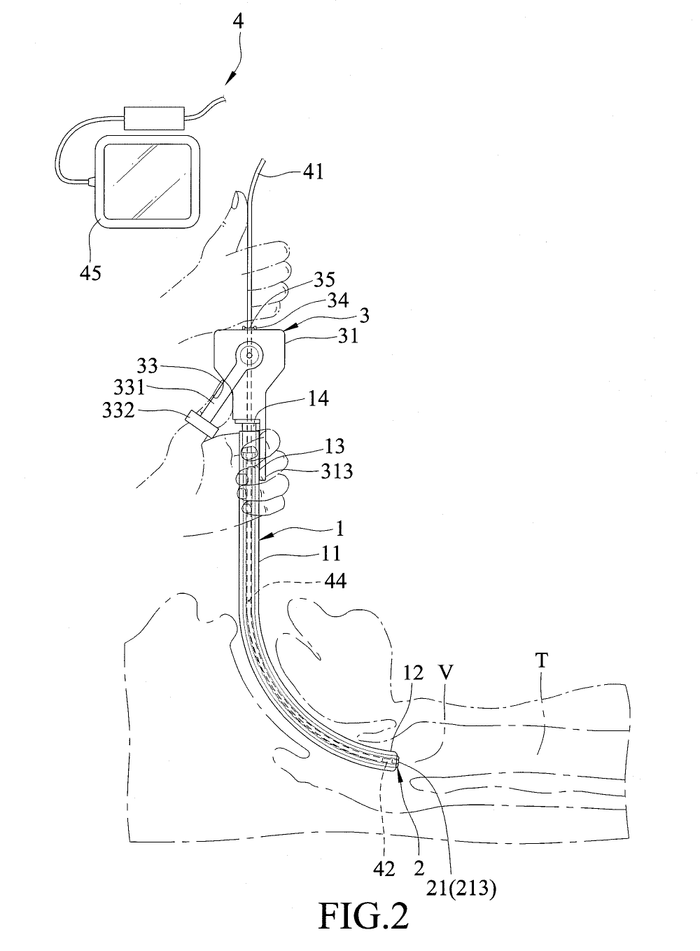

Referring to FIG. 2, the embodiment of an endotracheal intubation assistance apparatus according to this invention is adapted to assist in insertion of an endotracheal tube 1 into the trachea (T) of a patient. The endotracheal tube 1 includes a tube body 11 having a predetermined curvature and flexibility, a distal end 12, a proximal end 13, and a connector 14 sleeved on the proximal end 13. Alternatively, the connector 14 may be omitted from the endotracheal tube 1. The endotracheal intubation assistance apparatus includes a movable tubular stylet 2, a graspable controller 3, and a viewing device 4.

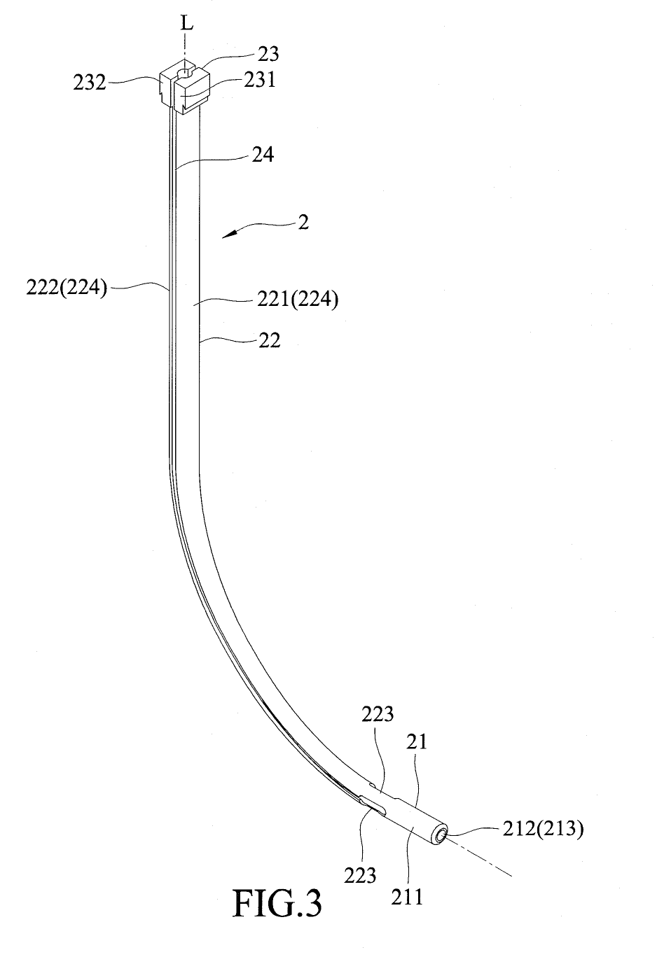

With further reference to FIG. 3, the movable tubular stylet 2 is flexible, and has a leading section 21, a tail section 23, a body section 22 connected between the leading section 21 and the tail section 23 along the length (L) of the movable tubular stylet 2, and two slits 24 extending through the body section 22 and the tail section 23. The movable tubular stylet 2 may be formed from a plastic material (such as Teflon) as one piece. The leading section 21 and the body section 22 are adapted to extend in the tube body 11 of the endotracheal tube 1. The leading section 21 is configured as a cylinder 211 having an inner bore 212, and a leading end 213 extendable outwardly from the distal end 12 of the endotracheal tube 1 and having a chamfered outer periphery. The body section 22 is divided by the slits 24 into a first strip 221 and a second strip 222. Each of the first and second strips 221, 222 has a weakened area 223 adjacent to the leading section 21, and an action area 224 disposed between the weakened area 223 and the tail section 23 and having a cross sectional area greater than that of the weakened area 223.

The tail section 23 is divided by the slits 24 into a first driven sheet 231 connected to the first strip 221, and a second driven sheet 232 connected to the second strip 222. In this embodiment, the first and second driven sheets 231, 232 of the tail section 23 are formed respectively and integrally with the first and second strips 221, 222 of the body section 22.

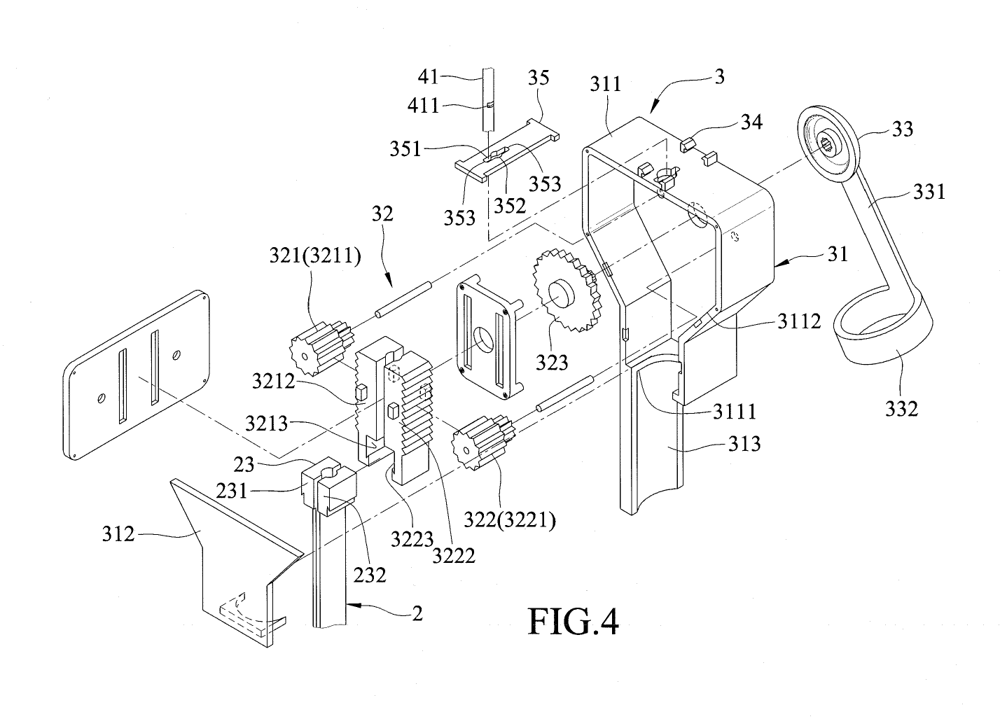

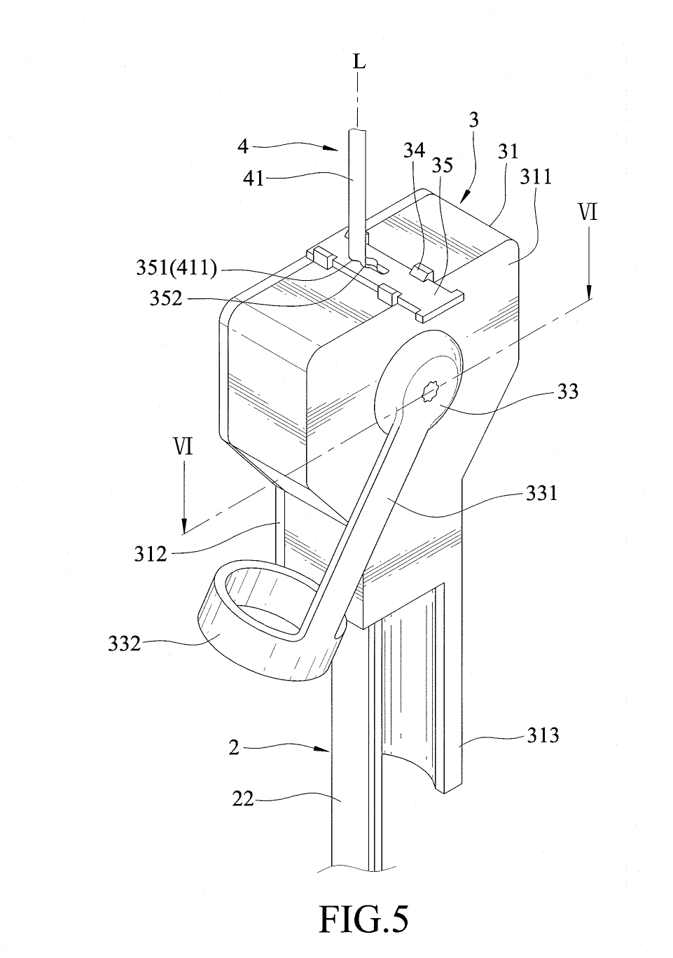

With further reference to FIGS. 4 and 5, the graspable controller 3 includes a main body 31, a driving mechanism 32 disposed in the main body 31, an operating member 33 operable for activating the driving mechanism 32, a track 34 disposed on the main body 31, and a position limiting member 35 mounted to the main body 31.

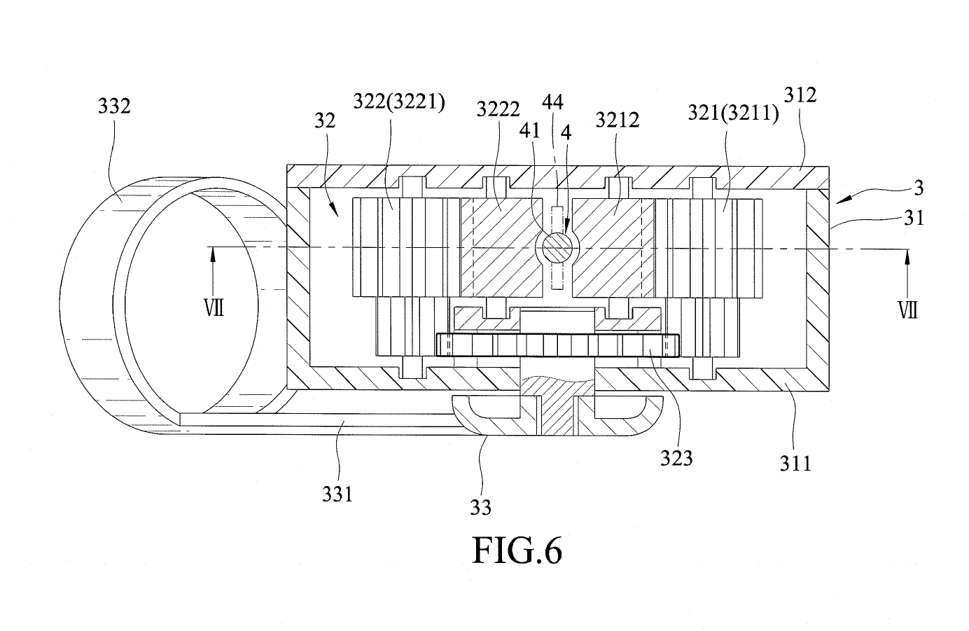

With particular reference to FIGS. 4, 6, and 7, the main body 3 includes a shell 311, a cover plate 312, and a handle 313 connected to and extending downwardly from the shell 311. The shell 311 has a lower end opening 3111 permitting the proximate end 13 of the endotracheal tube 1 to be sleeved thereon and permitting the tail section 23 of the movable tubular stylet 2 to extend thereinto, and a lateral side opening 3112 in spatial communication with the lower end opening 3111. The cover plate 312 covers openably the lateral side opening 3112. In this embodiment, the cover plate 312 is connected to the shell 311 by a tongue-and-groove engagement so as to cover the lateral side opening 3112.

The driving mechanism 32 is disposed for driving relative movement of the first and second driven sheets 231, 232 along the length (L), and includes a first connecting unit 321 connected to the first driven sheet 231, a second connecting unit 322 connected to the second driven sheet 232, and a primary gear 323 disposed pivotally on the shell 311 of the main body 31 for driving at least one of the first and second connecting units 321, 322. The first and second connecting units 321, 322 are located respectively at two sides of the primary gear 323 such that the first and second driven sheets 231, 232 move in opposite directions at a time.

The first connecting unit 321 includes a first gear 3211 disposed pivotally on the shell 311 of the main body 31 and meshing with the primary gear 323, and a first rack 3212 disposed movably in the shell 311 of the main body 31 and meshing with the first gear 3211 for driving the first driven sheet 231. The second connecting unit 322 includes a second gear 3221 disposed pivotally on the shell 311 of the main body 31 and meshing with the primary gear 323, and a second rack 3222 disposed movably in the shell 311 of the main body 31 and meshing with the second gear 3221 for driving the second driven sheet 232. The first rack 3212 is made of metal, and has a first dovetail groove 3213 engaging fittingly a portion of the first driven sheet 231. The second rack 3222 is made of metal, and has a second dovetail groove 3223 engaging fittingly a portion of the second driven sheet 232. During assembly, the cover plate 312 is connected to the shell 311 of the main body 31 after the first and second driven sheets 231, 232 are engaged respectively into the first and second dovetail grooves 3213, 3223 in the first and second racks 3212, 3222 and after the tail section 23 of the movable tubular stylet 2 is moved into the lower end opening 3111 in the shell 311, so as to prevent removal of the first and second driven sheets 231, 232 from the first and second dovetail grooves 3213, 3223 in the first and second racks 3212, 3222 and removal of the tail section 23 of the movable tubular stylet 2 from the lower end opening 3111 in the shell 311. As such, the movable tubular stylet 2 is convenient to replace.

The operating member 33 is connected to the primary gear 323 for driving rotation of the primary gear 323 in two opposite directions, is located outside the shell 311, and includes a connecting rod 331, and a finger sleeve 332 disposed on one end of said connecting rod 331 for extension of a finger of the user. The other end of said connecting rod 331 is connected to the primary gear 323.

The position limiting member 35 is configured as a tongue plate disposed movably on the track 34 for extension of the viewing device 4, and is formed with an engaging hole 351 having a central circular hole portion or release hole portion 352, and two locking hole portions 353 extending respectively from two opposite sides of the release hole portion 352 away from each other.

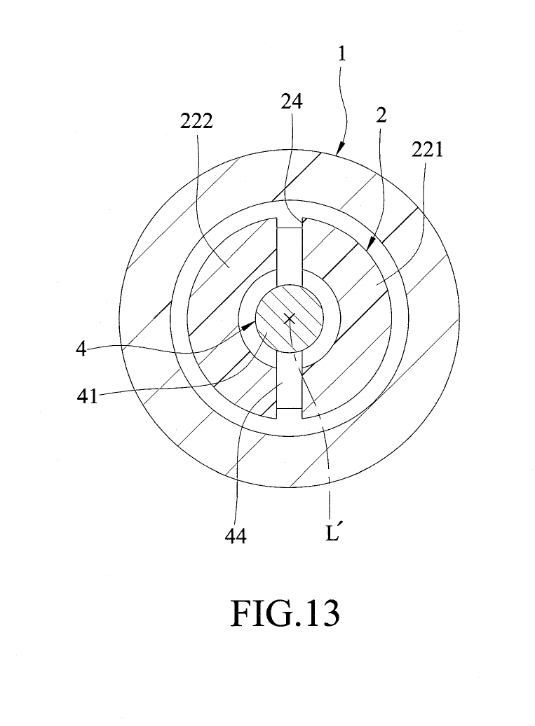

With particular reference to FIGS. 5, 7, and 8, the viewing device 4 includes a flexible elongate body 41 having a diameter of about 4-5 mm, a viewing head 42 disposed on a front end of the elongate body 41 and having a length of about 10 mm, a multi-joint link 43 disposed in the elongate body 41, and a pin 44 extending through the elongate body 41 and having two opposite ends extending respectively and outwardly from and movable along the slits 24, and a display 45 (see FIG. 2) electrically connected to the elongate body 41. The elongate body 41 extends through the engaging hole 351 in the position limiting member 35, and has a shrunk portion 411 disposed above the pin 44. The shrunk portion 411 has a cross sectional area smaller than that of the remaining portion of the elongate body 41. The viewing head 42 includes a small video camera 421 disposed therein, and at least one light-emitting member 422 disposed thereon. Alternatively, the viewing device 4 may be an optical-fiber viewing device. The multi-joint link 43 has a plurality of joint bodies 431 that are interconnected in series and that have rotating axes generally parallel to each other. Any two adjacent joint bodies 431 cannot twist relative to each other. As such, the elongate body 41 can only drive the viewing head 42 to swing upwardly and downwardly, such that the viewing head 42 cannot twist or swing leftwardly and rightwardly, thereby eliminating the disorientation problem occurred in the prior art. The pin 44 is parallel to the rotating axes. Since the pin 44 is movable along the slits 24, as shown in FIG. 13, rotation of the movable tubular stylet 2 about its central axis (L′) can be prevented, so as to promote orientation accuracy of the viewing head 42.

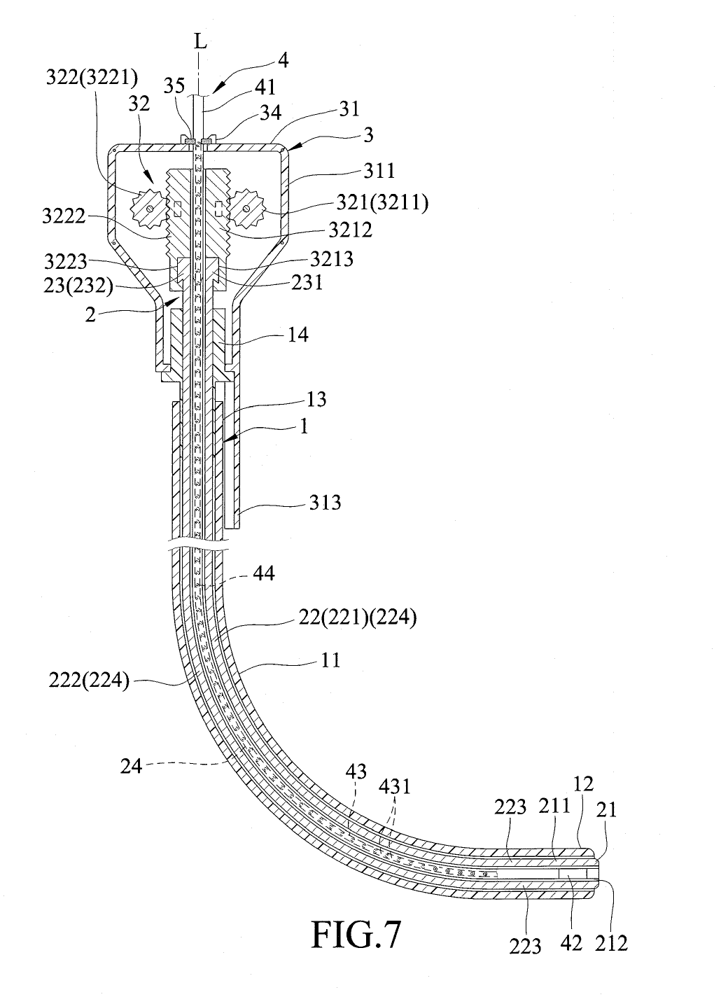

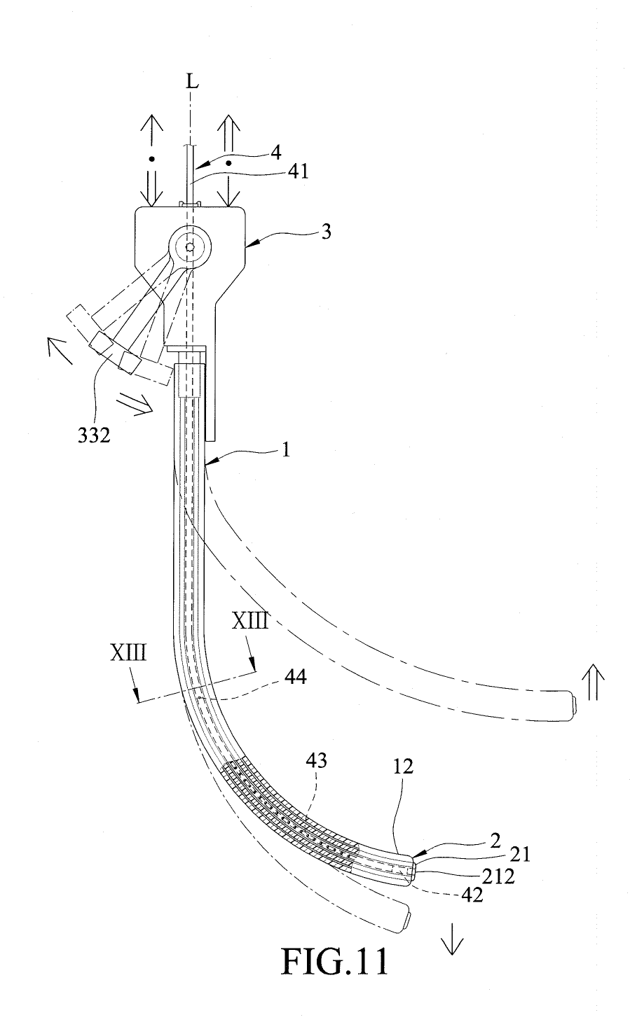

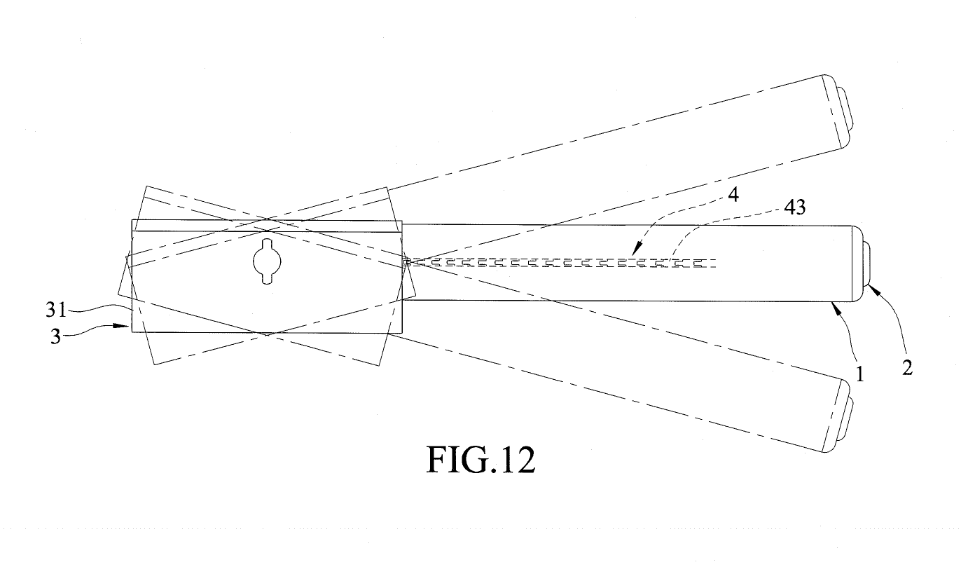

With particular reference to FIGS. 2, 7, and 11, an assembly of the elongate body 41 and the viewing head 42 is movable along the length (L) of the movable tubular stylet 2, and extends through the graspable controller 3 and out of the inner bore 212 in the leading section 21. When the finger sleeve 332 is swung in a downward direction indicated by a double arrow in FIG. 11, the first and second driven sheets 231, 232 move relative to each other along the length (L) of the movable tubular stylet 2, so as to transmit a force between the action areas 224 and the weakened areas 223 (see FIG. 7), thereby allowing the leading section 21 to drive synchronously the distal end 12 of the endotracheal tube 1 and the viewing head 42 to swing upwardly. When the finger sleeve 332 is swung in an upward direction indicated by a single arrow in FIG. 11, the first and second driven sheets 231, 232 move relative to each other along the length (L) of the movable tubular stylet 2, so as to transmit a force between the action areas 224 and the weakened areas 223 (see FIG. 7), thereby allowing the leading section 21 to drive synchronously the distal end 12 of the endotracheal tube 1 and the viewing head 42 to swing downwardly. The cylinder 211 of the leading section 21 is sufficiently stiff to support the viewing device 4 and the endotracheal tube 1, and the weakened areas 223 have an increased flexibility so as to allow the leading section 21 to swing easily. With further reference to FIG. 12, with inclusion of the multi-joint link 43 in the viewing device 4, when the main body 31 is rotated, the endotracheal tube 1 and the movable tubular stylet 2 co-rotate therewith.

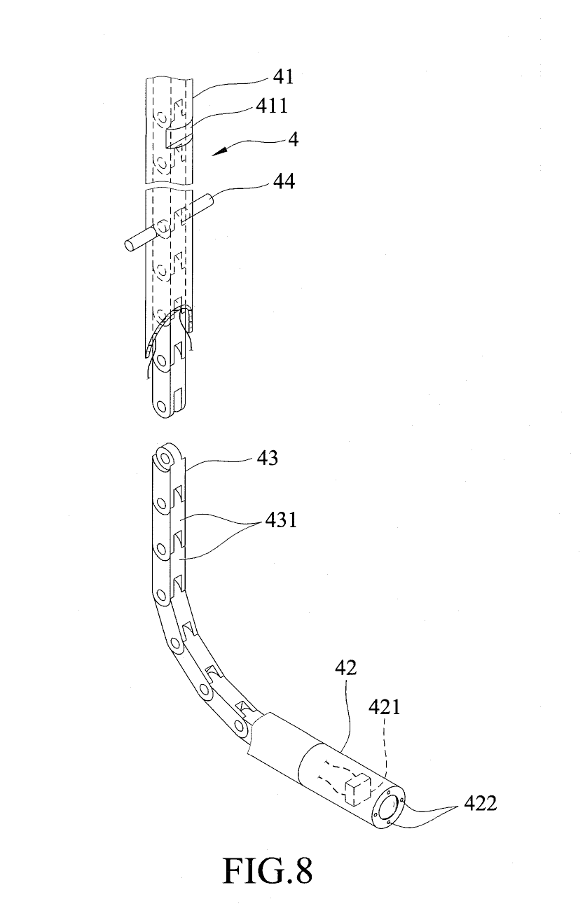

With particular reference to FIGS. 7, 9, and 10, during use, the position limiting member 35 is movable relative to the elongate body 41 between a release position and a locking position. At the release position, the shrunk portion 411 is spaced apart from the locking hole portions 353 of the engaging hole 351, so as to allow the elongate body 41 to move relative to the engaging hole 351. At the locking position, the shrunk portion 411 is engaged into one of the locking hole portions 353 to lock the elongate body 41 on the position limiting member 35, so as to prevent the elongate body 41 from rotation and movement along the length (L) of the movable tubular stylet 2, thereby allowing the viewing head 42 to be disposed entirely within the leading section 21 for avoiding the viewing head 42 (see FIG. 8) from being contaminated by saliva.

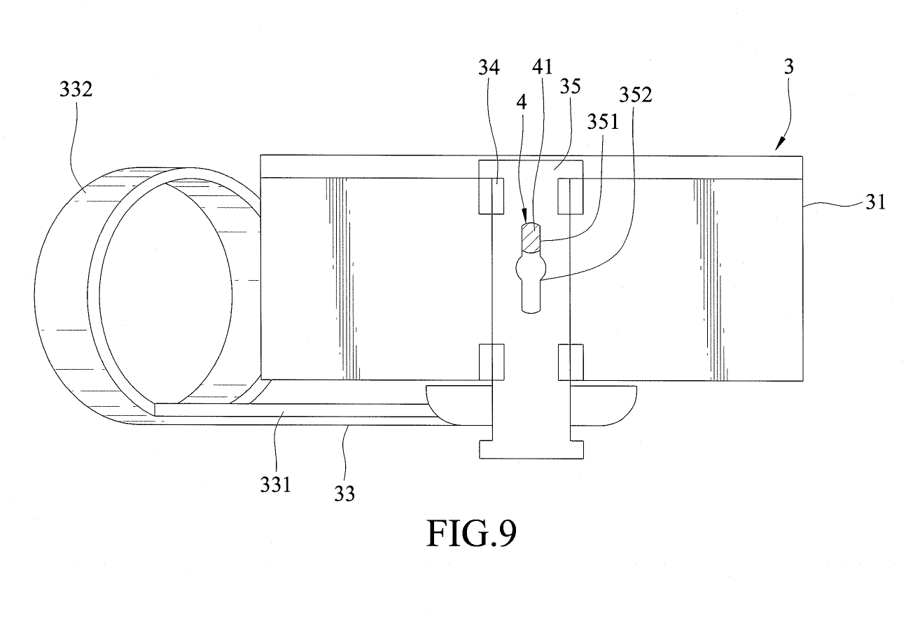

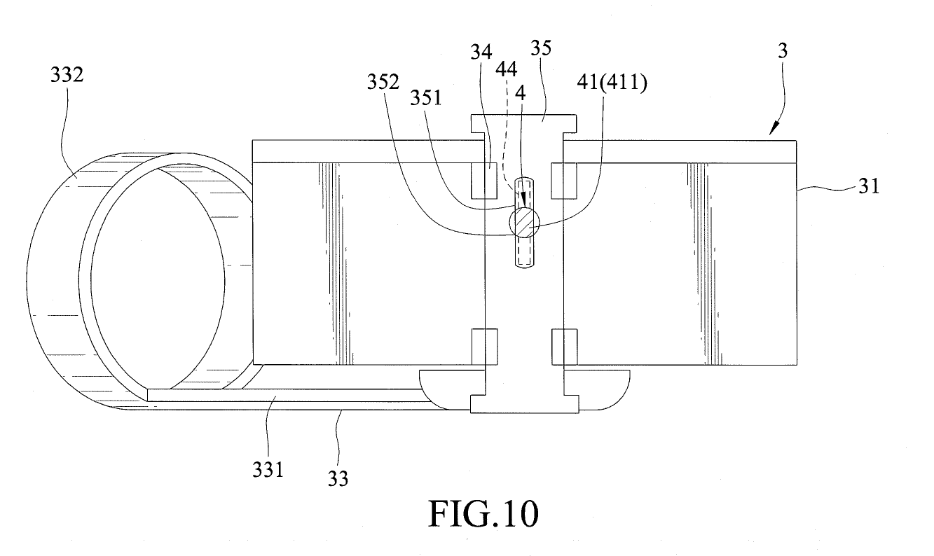

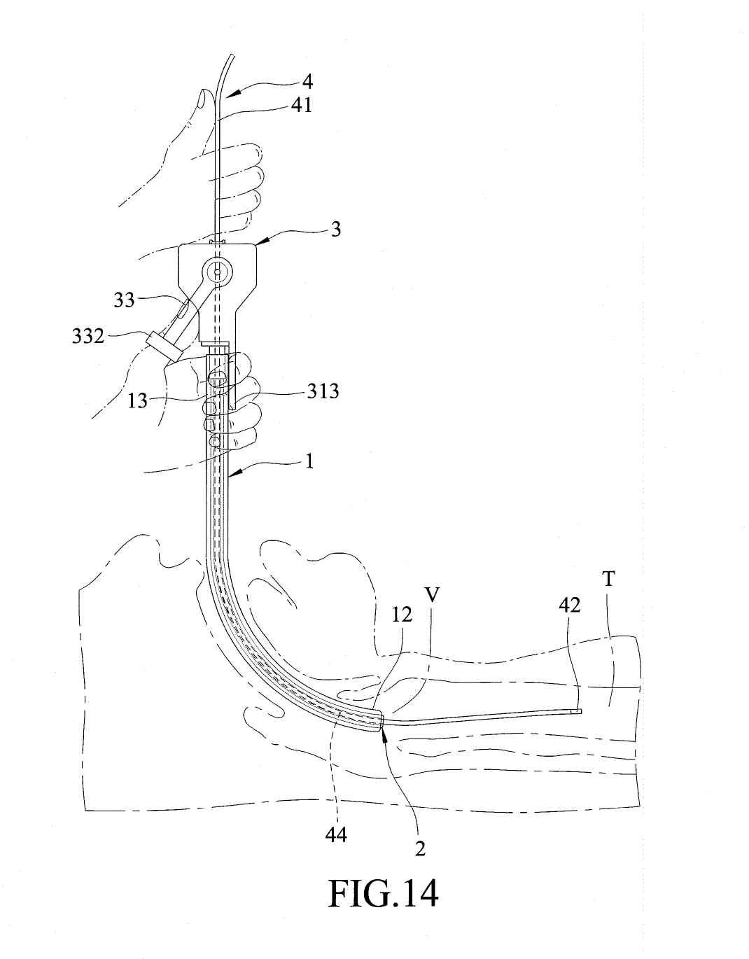

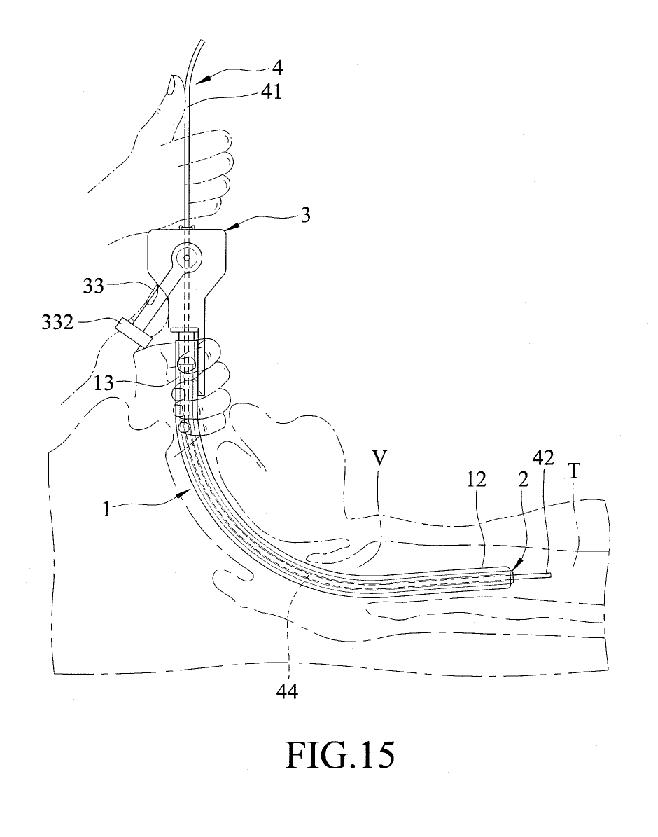

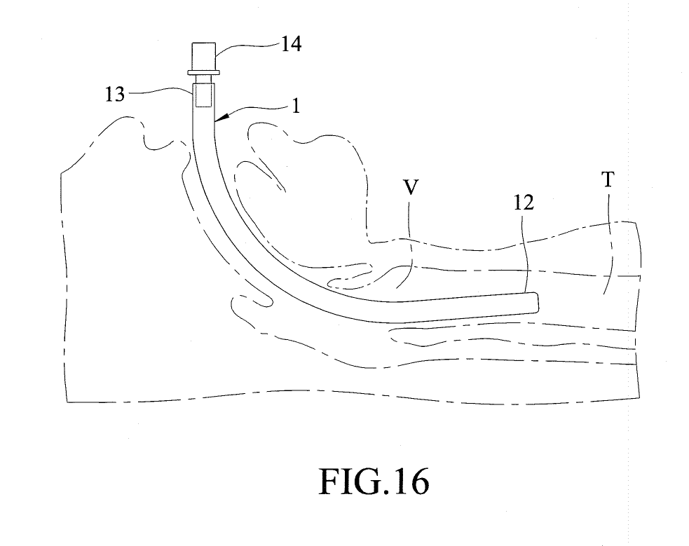

With particular reference to FIGS. 2 and 9, when it is desired to insert the endotracheal tube 1 into the trachea (T) of the patient, the position limiting member 35 is first placed at the locking position. Next, a thumb of one hand of the user is inserted into the finger sleeve 332, and the remaining fingers of the one hand of the user hold the handle 313. Afterwards, as shown in FIG. 11, the operating member 33 is operated using the thumb to control the movable tubular stylet 2 to swing the distal end 12 of the endotracheal tube 1 and the viewing head 42 of the viewing device 4 upwardly or downwardly. If necessary, as shown in FIG. 12, the graspable controller 3 can be operated using the wrist to swing the endotracheal tube 1 and the viewing head 42 leftwardly and rightwardly. As soon as the display 45 shows that the viewing head 42 is aligned with the vocal cords (V) (see FIG. 14), the position limiting member 35 is moved to the release position, as shown in FIG. 10, and the elongate body 41 is pushed using the other hand to move toward the trachea (T) by a predetermined distance, as shown in FIG. 14. At this time, the endotracheal tube 1 and the movable tubular stylet 2 are pushed into the trachea (T) along the elongate body 41, as shown in FIG. 15. Finally, the movable tubular stylet 2 and the elongate body 41 are drawn out of the endotracheal tube 1 so that the endotracheal tube 1 is left in the trachea (T), thereby completing the endotracheal intubation operation, as shown in FIG. 16.

With particular reference to FIG. 7, alternatively, the second gear 3221 may be omitted from the second connecting unit 322. In this case, the second rack 3222 is secured to the shell 311, so that the first driven sheet 231 can be moved relative to the second driven sheet 232 along the length (L) by the first rack 3212 and the first gear 3211 to drive upward and downward swinging movement of the leading section 21 of the movable tubular stylet 2. Conversely, the first gear 3211 may be omitted from the first connecting unit 321. In this case, the first rack 3212 is secured to the shell 311, so that the second driven sheet 232 can be moved relative to the first driven sheet 231 along the length (L) by the second rack 3222 and the second gear 3221 to drive upward and downward swinging movement of the leading section 21 of the movable tubular stylet 2.

To sum up, the endotracheal intubation assistance apparatus of this invention has the following advantages:
1. Different from the above-mentioned conventional assembly of a movable stylet and a viewing device that cannot move relative to each other, according to this invention, the viewing device 4 can be moved through the graspable controller 3 and the movable tubular stylet 2, such that both allocation of the vocal cords (V) at the first stage and establishment of a guide into the trachea (T) at the second stage can be achieved with relative ease, thereby promoting effectively the success rate of endotracheal intubation and reducing intubation time period. In this manner, difficult clinic situations can be overcome, for example, limited mouth opening degree, limited neck motion, and narrow throat anatomy.
2. Since the movable tubular stylet 2 is formed from the plastic material as one piece, and is not formed integrally with the viewing device 4, an assembly of the movable tubular stylet 2 and the viewing device 4 is made at a low cost, is easy to maintain, repair, and sterilize, and can be designed to be disposable to eliminate the sterilization procedure and cost.
3. The graspable controller 3 is convenient to operate. This can reduce the training time, and can promote clinic applicability.
4. Through the design of the multi-joint link 43, when the medical personnel use an assembly of the endotracheal tube 1, the movable tubular stylet 2, and the viewing device 4 to find the vocal cords (V), the assembly of the endotracheal tube 1, the movable tubular stylet 2, and the viewing device 4 cannot twist or swing leftwardly or rightwardly due to interference of soft tissues. Hence, the disorientation problem encountered by the prior art is eliminated to increase convenience during operation.

With this invention thus explained, it is apparent that numerous modifications and variations can be made without departing from the scope and spirit of this invention. It is therefore intended that this invention be limited only as indicated by the appended claims.

I claim:

1. An endotracheal intubation assistance apparatus adapted for assisting in insertion of an endotracheal tube into the trachea of a patient, the endotracheal tube including a flexible tube body having a distal end, and a proximal end, said endotracheal intubation assistance apparatus comprising:
a movable tubular stylet being flexible and having a leading section, a tail section, a body section connected between said leading section and said tail section along length of said movable tubular stylet, and two slits extending through said body section and said tail section, said leading section being configured as a cylinder and having an inner bore, said body section being divided by said slits into a first strip and a second strip, said tail section being divided by said slits into a first driven sheet connected to said first strip, and a second driven sheet connected to said second strip, said leading section and said body section being adapted to extend in the tube body of the endotracheal tube;
a graspable controller including a main body, a driving mechanism disposed on said main body, and an operating member operable for activating said driving mechanism, said main body being adapted to be connected to said proximal end of said endotracheal tube and permitting said tail section of said movable tubular stylet to extend thereinto, said driving mechanism being drivable to move first and second driven sheets relative to each other along the length of said movable tubular stylet; and
a viewing device including a flexible elongate body, a viewing head disposed on a leading end of said elongate body, and a multi-joint link disposed in said elongate body, said elongate body and said viewing head being movable in said graspable controller and being adapted to be extendable outwardly from the inner bore in said leading section of said movable tubular stylet such that, when said first and second driven sheets are driven to move relative to each other along the length of said movable tubular stylet, said leading section can swing the distal end of the endotracheal tube and said viewing head synchronously therewith, said multi-joint link having a plurality of joint bodies that are interconnected in series and that have rotating axes generally parallel to each other.

2. The endotracheal intubation assistance apparatus as claimed in claim 1, wherein said graspable controller further includes a position limiting member disposed on said main body and operable for locking said elongate body relative to said main body.

3. The endotracheal intubation assistance apparatus as claimed in claim 2, wherein said graspable controller further includes a track disposed on said main body, said position limiting member being configured as a tongue plate disposed movably on said track and permitting said elongate body of said viewing device to extend therethrough, said tongue plate being formed with an engaging hole that extends therethrough and that has a release hole portion and a locking hole portion, said elongate body further having a shrunk portion movable into said engaging hole such that, when said shrunk portion is located in said engaging hole, said shrunk portion is movable between said release hole portion and said locking hole portion, said position limiting member being movable relative to said elongate body between a locking position whereat said shrunk portion is engaged within said locking hole portion of said engaging hole so as to prevent movement and rotation of said elongate body relative to said graspable controller, and a release position whereat said shrunk is disengaged from said locking hole portion so as to allow for movement and rotation of said elongate body relative to said graspable controller.

4. The endotracheal intubation assistance apparatus as claimed in claim 3, wherein, when said position limiting member is disposed at the locking position relative to said elongate body, said viewing head does not extend outwardly from said leading section.

5. The endotracheal intubation assistance apparatus as claimed in claim 1, wherein said viewing device further includes a pin extending through said elongate body and having two opposite ends that extend respectively and outwardly from said elongate body and that are disposed respectively and movably within said slits, said pin being parallel to the rotating axes.

6. The endotracheal intubation assistance apparatus as claimed in claim 5, wherein said graspable controller further includes a position limiting member disposed on said main body and operable for locking said elongate body relative to said main body.

7. The endotracheal intubation assistance apparatus as claimed in claim 6, wherein said graspable controller further includes a track disposed on said main body, said position limiting member being configured as a tongue plate disposed movably on said track and permitting said elongate body of said viewing device to extend therethrough, said tongue plate being formed with an engaging hole that extends therethrough and that has a release hole portion and a locking hole portion, said elongate body further having a shrunk portion movable into said engaging hole such that, when said shrunk portion is located in said engaging hole, said shrunk portion is movable between said release hole portion and said locking hole portion, said position limiting member being movable relative to said elongate body between a locking position whereat said shrunk portion is engaged within said locking hole portion of said engaging hole so as to prevent movement and rotation of said elongate body relative to said graspable controller, and a release position whereat said shrunk is disengaged from said locking hole portion so as to allow for movement and rotation of said elongate body relative to said graspable controller.

8. The endotracheal intubation assistance apparatus as claimed in claim 7, wherein, when said tongue plate is disposed at the locking position relative to said elongate body, said viewing head does not extend outwardly from said leading section.

9. The endotracheal intubation assistance apparatus as claimed in claim 1, wherein said driving mechanism of said graspable controller includes a first connecting unit connected to said first driven sheet, a second connecting unit connected to said second driven sheet, and a primary gear disposed pivotally to said main body for driving at least one of said first and second connecting units, said operating member being connected to said primary gear and operable for driving rotation of said primary gear in two opposite directions, said first and second connecting units being located respectively at two sides of said primary gear.

10. The endotracheal intubation assistance apparatus as claimed in claim 9, wherein said first connecting unit includes a first gear disposed pivotally on said main body and meshing with said primary gear, and a first rack disposed movably in said main body, meshing with said first gear, and connected co-movably to said first driven sheet, said second connecting unit including a second gear disposed pivotally on said main body and meshing with said primary gear, and a second rack disposed pivotally in said main body, meshing with said second gear, and connected co-movably to said second driven sheet.

11. The endotracheal intubation assistance apparatus as claimed in claim 10, wherein said main body of said graspable controller includes a shell and a cover plate, said shell having a lower end opening and a lateral side opening in spatial communication with said lower end opening, said cover plate covering openably said lateral side opening, said cover plate being openable such that, during assembly, said first and second driven sheets are connected respectively to said first and second racks, and said movable tubular stylet is moved into said lower end opening of said shell.

12. The endotracheal intubation assistance apparatus as claimed in claim 11, wherein said operating member is mounted to said shell, and includes a connecting rod, and a finger sleeve disposed on one end of said connecting rod, the other end of said connecting rod being connected to said primary gear.
